(19) United States
(12) Patent Application Publication
(10) Pub. No.: US 2025/0345361 A1
(43) Pub. Date: Nov. 13, 2025

(54) COMPOSITIONS AND METHODS TO IMPROVE CALF GROWTH AND REDUCE INCIDENCE OF SCOURS

(71) Applicant: ELANCO US INC., Greenfield, IN (US)

(72) Inventors: Sarah SCHULING, Greenfield, IN (US); Daniel SCHIMEK, Greenfield, IN (US); Liang CHEN, Greenfield, IN (US)

(21) Appl. No.: 18/869,665

(22) PCT Filed: Jun. 1, 2023
(86) PCT No.: PCT/US2023/024129

§ 371 Date: Nov. 26, 2024

Related U.S. Application Data
(60) Provisional application No. 63/347,627, filed on Jun. 1, 2022.

(51) Int. Cl. A61K 33/06; A23K 10/16; A23K 10/30; A23K 20/28; A61K 36/064; A61K 36/81; A61P 1/12

(52) U.S. Cl. CPC A61K 33/06; A23K 10/16; A23K 10/30; A23K 20/28; A61K 36/064; A61K 36/81; A61P 1/12

(57) ABSTRACT

The present disclosure relates to compositions comprising capsicum, functional mineral compounds, and yeast cell wall. Furthermore, methods of feeding the composition to animals such as bovines are provided to demonstrate beneficial effects on the animals. The compositions and methods described herein can benefit overall animal wellbeing and future productivity, especially in younger animals.

### CROSS-REFERENCE TO RELATED APPLICATIONS

This application claims the benefit under 35 USC § 119(e) of U.S. Provisional Application Ser. No. 63/347,627, filed on Jun. 1, 2022, the entire disclosure of which is incorporated herein by reference.

### FIELD OF THE INVENTION

The present disclosure relates to compositions comprising capsicum, functional mineral compounds, and yeast cell wall. Furthermore, methods of feeding the composition to animals such as bovines are provided to demonstrate beneficial effects on the animals. The compositions and methods described herein can benefit overall animal wellbeing and future productivity, especially in younger animals.

### BACKGROUND

Control of animal diseases is an important consideration for the agriculture and food production industries. In modern commercial operations, steps to protect animals of every age can be undertaken in order to avoid disease outbreaks and to promote the overall health of the animal population.

For instance, in commercial dairy cattle operations in the United States, dairy calves are generally removed from dams and the calves are raised in an individual or a small group housing system in order to minimize or prevent the spread of disease from dam to calf in the early life of the calf. During this stressful phase of life, the ability to maintain healthy calves with better growth performance is typically beneficial to overall animal wellbeing and future productivity. Thus, there exists a need for new compositions and methods to assist with management of young animals in order to promote development and prevent disease incidence.

### SUMMARY

The present disclosure provides compositions comprising capsicum, functional mineral compounds, and yeast cell wall as well as associated methods. The compositions and methods of the present disclosure surprisingly result to a consistent improvement in growth measurements and a reduction in gastrointestinal disease incidences in animals such as young calves. The present disclosure describes compositions comprising capsicum, a functional mineral compound such as bentonite, and a yeast cell wall (i.e., C+FMC+YCW) that can improve growth performance and reduce scour events when fed to cattle. In contrast, combining two of the ingredients (i.e., two of capsicum, a functional mineral compound, or a yeast cell wall) was ineffective compared to the compositions comprising capsicum, a functional mineral compound such as bentonite, and a yeast cell wall.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 shows average daily gain (ADG) from arrival through weaning in calves fed compositions of the present disclosure. Values a,b with different superscripts differ P<0.05.

FIG. 2 shows increase of body weight (BW) by week in calves fed compositions of the present disclosure. Values a,b with different superscripts differ P<0.05.

FIG. 3 shows distribution of TNF-α (log 10) plasma concentration by dietary treatment group and scour event in calves fed compositions of the present disclosure. (0=no scour events; 1=scour event).

FIG. 4 shows average daily starter intake by week in calves fed compositions of the present disclosure.

FIG. 5 shows average daily gain (ADG) over two weighing intervals in calves fed compositions of the present disclosure.

FIG. 6 shows increase of body weight (BW) by week in calves fed compositions of the present disclosure.

FIG. 7 shows average daily gain (ADG) at four different weeks of age in calves fed compositions of the present disclosure.

FIG. 8 shows increase of body weight (BW) at five different weeks of age in calves fed compositions of the present disclosure.

FIG. 9 shows an increase in dry feed intake at different weeks of age in calves fed compositions of the present disclosure. Values a,b with different superscripts differ P<0.05.

FIG. 10 shows incidence of scours in calves fed compositions of the present disclosure. Values a,b with different superscripts differ P<0.05.

### DETAILED DESCRIPTION

In an illustrative aspect, a composition comprising capsicum, a functional mineral compound, and a yeast cell wall is provided. In an embodiment, the functional mineral compound comprises an aluminum phyllosilicate clay. In an embodiment, the functional mineral compound consists essentially of an aluminum phyllosilicate clay (e.g., greater than 95 wt. % or even greater than 99 wt. %). In an embodiment, the functional mineral compound consists of an aluminum phyllosilicate clay. In an embodiment, the functional mineral compound comprises bentonite. In an embodiment, the functional mineral compound consists essentially of bentonite. In an embodiment, the functional mineral compound consists of bentonite. Aluminum phyllosilicate clays and bentonite include those described in, for instance, U.S. Patent Publication No. 2017/0095508, U.S. Patent Publication No. 2022/0338504, and U.S. Patent Publication No. 2022/0142204, each of which is incorporated by reference in their entirety.

In an embodiment, the capsicum is present in the composition between about 1 mg to about 1000 mg. In an embodiment, the capsicum is present in the composition between about 50 mg to about 750 mg. In an embodiment, the capsicum is present in the composition between about 100 mg to about 500 mg. In an embodiment, the capsicum is present in the composition at about 100 mg. In an embodiment, the capsicum is present in the composition at about 200 mg. In an embodiment, the capsicum is present in the composition at about 300 mg. In an embodiment, the capsicum is present in the composition at about 400 mg. In an embodiment, the capsicum is present in the composition at about 500 mg. In an embodiment, the capsicum is present in the composition at about 600 mg. In an embodiment, the capsicum is present in the composition at about 700 mg. In an embodiment, the capsicum is present in the composition at about 800 mg. In an embodiment, the capsicum is present in the composition at about 900 mg. In an embodiment, the capsicum is present in the composition at about 1000 mg.

In an embodiment, the amount of capsicum administered is between about 1 mg/day/animal to about 1000 mg/day/animal. In an embodiment, the amount of capsicum administered is between about 50 mg/day/animal to about 750 mg/day/animal. In an embodiment, the amount of capsicum administered is between about 100 mg/day/animal to about 500 mg/day/animal.

In an embodiment, the amount of capsicum that is administered is between about 1 mg/day/animal to about 1000 mg/day/animal. In an embodiment, the amount of capsicum that is administered is between about 50 mg/day/animal to about 750 mg/day/animal. In an embodiment, the amount of capsicum that is administered is between about 100 mg/day/animal to about 500 mg/day/animal.

In an embodiment, the functional mineral compound is present in the composition between about 1 g to about 10 g. In an embodiment, the functional mineral compound is present in the composition between about 1 g to about 5 g. In an embodiment, the functional mineral compound is present in the composition at about 1 g. In an embodiment, the functional mineral compound is present in the composition at about 2 g. In an embodiment, the functional mineral compound is present in the composition at about 3 g. In an embodiment, the functional mineral compound is present in the composition at about 4 g. In an embodiment, the functional mineral compound is present in the composition at about 5 g. In an embodiment, the functional mineral compound is present in the composition at about 6 g. In an embodiment, the functional mineral compound is present in the composition at about 7 g. In an embodiment, the functional mineral compound is present in the composition at about 8 g. In an embodiment, the functional mineral compound is present in the composition at about 9 g. In an embodiment, the functional mineral compound is present in the composition at about 10 g.

In an embodiment, the amount of the functional mineral compound that is administered is between about 1 g/day/animal to about 10 g/day/animal. In an embodiment, the amount of the functional mineral compound that is administered is between about 1 g/day/animal to about 5 g/day/animal.

In an embodiment, the yeast cell wall is present in the composition between about 1 g to about 10 g. In an embodiment, the yeast cell wall is present in the composition between about 1 g to about 5 g. In an embodiment, the yeast cell wall is present in the composition at about 1 g. In an embodiment, the yeast cell wall is present in the composition at about 2 g. In an embodiment, the yeast cell wall is present in the composition at about 3 g. In an embodiment, the yeast cell wall is present in the composition at about 4 g. In an embodiment, the yeast cell wall is present in the composition at about 5 g. In an embodiment, the yeast cell wall is present in the composition at about 6 g. In an embodiment, the yeast cell wall is present in the composition at about 7 g. In an embodiment, the yeast cell wall is present in the composition at about 8 g. In an embodiment, the yeast cell wall is present in the composition at about 9 g. In an embodiment, the yeast cell wall is present in the composition at about 10 g.

In an embodiment, the amount of the yeast cell wall that is administered is between about 1 g/day/animal to about 10 g/day/animal. In an embodiment, the amount of the yeast cell wall that is administered is between about 1 g/day/animal to about 5 g/day/animal.

As used herein, the capsicum may be added to feed at a concentration of about 0.01 lb/ton feed or more, about 0.05 lb/ton feed or more, about 0.1 lb/ton feed or more, about 0.2 lb/ton feed or more, about 0.3 lb/ton feed or more, about 0.4 lb/ton feed or more, or about 0.5 lb/ton feed or more. For example, the capsicum concentration can be from about 0.01 lb/ton to about 1 lb/ton feed, from about 0.1 lb/ton to about 1 lb/ton feed, or from about 0.1 lb/ton to about 0.5 lb/ton feed. Capsicum may be selected from the group consisting of a capsaicinoid, a vanilloid, Capsicum, macerated hot peppers, ground hot peppers, hot pepper extract, capsaicin-containing plant materials, encapsulated ground peppers, a coated capsaicin product, and combinations of any thereof. In an embodiment, the capsicum is encapsulated ground peppers. The capsicum comprises about 45% to about 55% ground peppers and/or comprises about 0.4% to about 0.6% capsaicinoids.

The yeast cell wall can include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae.

In an embodiment, the capsicum, the functional mineral compound, and the yeast cell wall are present at a weight ratio of C:FMC:YCW. In an embodiment, the weight ratio of C:FMC:YCW is 1-1000 mg:1-10 g:1-10 g, 100-1000 mg:1-10 g:1-10 g, or 100-500 mg:1-10 g:1-10 g. In an embodiment, the ratio of C:FMC:YCW is 100 mg:5 g:2 g. In an embodiment, the ratio of C:FMC:YCW is 300 mg:5 g:2 g. In an embodiment, the ratio of C:FMC:YCW is 500 mg:5 g:2 g. In an embodiment, the ratio of C:FMC:YCW is 100 mg:3 g:2 g. In an embodiment, the ratio of C:FMC:YCW is 300 mg:3 g:2 g. In an embodiment, the ratio of C:FMC:YCW is 500 mg:3 g:2 g.

In an illustrative aspect, a formulation is provided. The formulation comprises i) a composition comprising capsicum, a functional mineral compound, and a yeast cell wall and ii) an animal food. In an embodiment, the animal food is a dry animal food. In an embodiment, the dry animal food is a starter grain. In an embodiment, the dry animal food is a texturized animal food. In an embodiment, the dry animal food is a calf starter animal food. In an embodiment, the dry animal food is a texturized calf starter animal food. Such dry animal foods are well known to the skilled artisan. In an embodiment, the animal food is a liquid animal food. In an embodiment, the liquid animal food is milk. The previously described embodiments of the composition are also applicable to the embodiments of the formulation described herein.

In an embodiment, the dry animal food is present in the formulation between about 0.5 lb to about 5 lbs. In an embodiment, the dry animal food is present in the formulation between about 0.5 lb to about 3 lbs. In an embodiment, the dry animal food is present in the formulation between about 0.5 lb to about 1 lb. In an embodiment, the dry animal food is present in the formulation between about 1 lb to about 1.5 lbs. In an embodiment, the dry animal food is present in the formulation between about 1.5 lb to about 2 lbs. In an embodiment, the dry animal food is present in the formulation between about 2 lb to about 2.5 lbs. In an embodiment, the dry animal food is present in the formulation between about 2.5 lb to about 3 lbs. In an embodiment, the dry animal food is present in the formulation between about 3 lb to about 3.5 lbs. In an embodiment, the dry animal food is present in the formulation between about 3.5 lb to about 4 lbs. In an embodiment, the dry animal food is present in the formulation between about 4 lb to about 4.5 lbs. In an embodiment, the dry animal food is present in the formulation between about 4.5 lb to about 5 lbs.

In an embodiment, the liquid animal food is present in the formulation between about 1 quart to about 5 quarts. In an embodiment, the liquid animal food is present in the formulation at about 1 quart. In an embodiment, the liquid animal food is present in the formulation at about 1.5 quarts. In an embodiment, the liquid animal food is present in the formulation at about 2 quarts. In an embodiment, the liquid animal food is present in the formulation at about 2.5 quarts. In an embodiment, the liquid animal food is present in the formulation at about 3 quarts. In an embodiment, the liquid animal food is present in the formulation at about 3.5 quarts. In an embodiment, the liquid animal food is present in the formulation at about 4 quarts. In an embodiment, the liquid animal food is present in the formulation at about 4.5 quarts. In an embodiment, the liquid animal food is present in the formulation at about 5 quarts.

In an illustrative aspect, a method of improving gastrointestinal health of an animal is provided. The method comprises the step of feeding the animal a composition comprising capsicum, a functional mineral compound, and a yeast cell wall product, wherein the composition improves gastrointestinal health in the animal. The previously described embodiments of the composition are also applicable to the methods of improving gastrointestinal health of an animal described herein. For instance, the composition utilized with the method can be any of the compositions described herein. Furthermore, the composition utilized with the method can be comprised in any of the formulations described herein.

In an embodiment, improving gastrointestinal health comprises a decrease in frequency of scour events by the animal. In an embodiment, improving gastrointestinal health comprises a decrease in medical intervention from scour events by the animal. Scour events, also known as scours, are well known in the art, for instance diarrhea by an animal such as a young animal (e.g., calf).

In an embodiment, improving gastrointestinal health comprises a decrease in concentration of Tumor necrosis factor alpha (TNF-α) in plasma of the animal. In an embodiment, improving gastrointestinal health comprises an improved innate immune response in the animal.

In an embodiment, the animal is a ruminant animal. In an embodiment, the ruminant animal is selected from the group consisting of cattle, goats, sheep, giraffes, American Bison, European bison, yaks, water buffalo, deer, camels, alpacas, llamas, wildebeest, antelope, pronghorn, and nilgai. In an embodiment, the ruminant animal is raised for meat production. In an embodiment, the ruminant animal is raised for dairy production. In an embodiment, the ruminant animal is selected from the group consisting of cattle, buffalo, sheep, and goats. In an embodiment, the ruminant animal is a bovine. In an embodiment, the ruminant animal is a buffalo. In an embodiment, the ruminant animal is a sheep. In an embodiment, the ruminant animal is a goat. In an embodiment, the ruminant animal is a beef cattle. In an embodiment, the ruminant animal is a dairy cattle. In an embodiment, the ruminant animal is a calf. In an embodiment, the calf is a pre-weaned calf. In an embodiment, the ruminant animal is a male. In an embodiment, the ruminant animal is a female.

In an embodiment, the composition is administered to the animal one time per day. In an embodiment, the composition is administered to the animal two times per day. In an embodiment, the composition is administered to the animal three times per day.

In an illustrative aspect, a method of increasing growth performance of an animal is provided. The method comprises the step of feeding the animal a composition comprising capsicum, a functional mineral compound, and a yeast cell wall product, wherein the composition increases growth performance of the animal. The previously described embodiments of the composition are also applicable to the methods of increasing growth performance of an animal described herein.

In an embodiment, increasing growth performance comprises an increase in average daily gain (ADG) of the animal. Means of calculating ADG of an animal are well known to the skilled artisan. In an embodiment, the increase in ADG of the animal is evaluated at weaning of the animal. In an embodiment, the increase in ADG of the animal is evaluated post-weaning of the animal.

In an illustrative aspect, a method of increasing body weight of an animal is provided. The method comprises the step of feeding the animal a composition comprising capsicum, a functional mineral compound, and a yeast cell wall product, wherein the composition increases body weight of the animal. The previously described embodiments of the composition are also applicable to the methods of increasing body weight of an animal described herein. In an embodiment, the increase in body weight of the animal is evaluated at weaning of the animal. In an embodiment, the increase in body weight of the animal is evaluated post-weaning of the animal. Means of calculating body weight of an animal are well known to the skilled artisan.

In an illustrative aspect, a method of improving intake of food by an animal is provided. The method comprises the step of feeding the animal a composition comprising capsicum, a functional mineral compound, and a yeast cell wall product, wherein the composition improves intake of food by the animal. The previously described embodiments of the composition are also applicable to the methods of improving intake of food by an animal described herein. In an embodiment, the food is a dry food. In an embodiment, the dry food is a starter grain. In an embodiment, the dry food is a texturized animal food. In an embodiment, the dry food is a calf starter animal food. In an embodiment, the dry food is a texturized calf starter animal food. In an embodiment, the food is a liquid food. In an embodiment, the liquid food is milk.

The following numbered embodiments are contemplated and are non-limiting:
1. A composition comprising capsicum, a functional mineral compound, and a yeast cell wall.
2. The composition of clause 1, any other suitable clause, or any combination of suitable clauses, wherein the functional mineral compound comprises an aluminum phyllosilicate clay.
3. The composition of clause 1, any other suitable clause, or any combination of suitable clauses, wherein the functional mineral compound consists essentially of an aluminum phyllosilicate clay.
4. The composition of clause 1, any other suitable clause, or any combination of suitable clauses, wherein the functional mineral compound consists of an aluminum phyllosilicate clay.
5. The composition of clause 1, any other suitable clause, or any combination of suitable clauses, wherein the functional mineral compound comprises bentonite.
6. The composition of clause 1, any other suitable clause, or any combination of suitable clauses, wherein the functional mineral compound consists essentially of bentonite.
7. The composition of clause 1, any other suitable clause, or any combination of suitable clauses, wherein the functional mineral compound consists of bentonite.
8. The composition of clause 1, any other suitable clause, or any combination of suitable clauses, wherein the capsicum is present in the composition between about 1 mg to about 1000 mg.
9. The composition of clause 1, any other suitable clause, or any combination of suitable clauses, wherein the capsicum is present in the composition between about 50 mg to about 750 mg.
10. The composition of clause 1, any other suitable clause, or any combination of suitable clauses, wherein the capsicum is present in the composition between about 100 mg to about 500 mg.
11. The composition of clause 1, any other suitable clause, or any combination of suitable clauses, wherein the capsicum is present in the composition at about 100 mg.
12. The composition of clause 1, any other suitable clause, or any combination of suitable clauses, wherein the capsicum is present in the composition at about 200 mg.
13. The composition of clause 1, any other suitable clause, or any combination of suitable clauses, wherein the capsicum is present in the composition at about 300 mg.
14. The composition of clause 1, any other suitable clause, or any combination of suitable clauses, wherein the capsicum is present in the composition at about 400 mg.
15. The composition of clause 1, any other suitable clause, or any combination of suitable clauses, wherein the capsicum is present in the composition at about 500 mg.
16. The composition of clause 1, any other suitable clause, or any combination of suitable clauses, wherein the capsicum is present in the composition at about 600 mg.
17. The composition of clause 1, any other suitable clause, or any combination of suitable clauses, wherein the capsicum is present in the composition at about 700 mg.
18. The composition of clause 1, any other suitable clause, or any combination of suitable clauses, wherein the capsicum is present in the composition at about 800 mg.
19. The composition of clause 1, any other suitable clause, or any combination of suitable clauses, wherein the capsicum is present in the composition at about 900 mg.
20. The composition of clause 1, any other suitable clause, or any combination of suitable clauses, wherein the capsicum is present in the composition at about 1000 mg.
21. The composition of clause 1, any other suitable clause, or any combination of suitable clauses, wherein the functional mineral compound is present in the composition between about 1 g to about 10 g.
22. The composition of clause 1, any other suitable clause, or any combination of suitable clauses, wherein the functional mineral compound is present in the composition between about 1 g to about 5 g.
23. The composition of clause 1, any other suitable clause, or any combination of suitable clauses, wherein the functional mineral compound is present in the composition at about 1 g.
24. The composition of clause 1, any other suitable clause, or any combination of suitable clauses, wherein the functional mineral compound is present in the composition at about 2 g.
25. The composition of clause 1, any other suitable clause, or any combination of suitable clauses, wherein the functional mineral compound is present in the composition at about 3 g.
26. The composition of clause 1, any other suitable clause, or any combination of suitable clauses, wherein the functional mineral compound is present in the composition at about 4 g.
27. The composition of clause 1, any other suitable clause, or any combination of suitable clauses, wherein the functional mineral compound is present in the composition at about 5 g.
28. The composition of clause 1, any other suitable clause, or any combination of suitable clauses, wherein the functional mineral compound is present in the composition at about 6 g.
29. The composition of clause 1, any other suitable clause, or any combination of suitable clauses, wherein the functional mineral compound is present in the composition at about 7 g.
30. The composition of clause 1, any other suitable clause, or any combination of suitable clauses, wherein the functional mineral compound is present in the composition at about 8 g.
31. The composition of clause 1, any other suitable clause, or any combination of suitable clauses, wherein the functional mineral compound is present in the composition at about 9 g.
32. The composition of clause 1, any other suitable clause, or any combination of suitable clauses, wherein the functional mineral compound is present in the composition at about 10 g.
33. The composition of clause 1, any other suitable clause, or any combination of suitable clauses, wherein the yeast cell wall is present in the composition between about 1 g to about 10 g.
34. The composition of clause 1, any other suitable clause, or any combination of suitable clauses, wherein the yeast cell wall is present in the composition between about 1 g to about 5 g.
35. The composition of clause 1, any other suitable clause, or any combination of suitable clauses, wherein the yeast cell wall is present in the composition at about 1 g.
36. The composition of clause 1, any other suitable clause, or any combination of suitable clauses, wherein the yeast cell wall is present in the composition at about 2 g.
37. The composition of clause 1, any other suitable clause, or any combination of suitable clauses, wherein the yeast cell wall is present in the composition at about 3 g.
38. The composition of clause 1, any other suitable clause, or any combination of suitable clauses, wherein the yeast cell wall is present in the composition at about 4 g.
39. The composition of clause 1, any other suitable clause, or any combination of suitable clauses, wherein the yeast cell wall is present in the composition at about 5 g.
40. The composition of clause 1, any other suitable clause, or any combination of suitable clauses, wherein the yeast cell wall is present in the composition at about 6 g.
41. The composition of clause 1, any other suitable clause, or any combination of suitable clauses, wherein the yeast cell wall is present in the composition at about 7 g.
42. The composition of clause 1, any other suitable clause, or any combination of suitable clauses, wherein the yeast cell wall is present in the composition at about 8 g.
43. The composition of clause 1, any other suitable clause, or any combination of suitable clauses, wherein the yeast cell wall is present in the composition at about 9 g.
44. The composition of clause 1, any other suitable clause, or any combination of suitable clauses, wherein the yeast cell wall is present in the composition at about 10 g.
45. The composition of clause 1, any other suitable clause, or any combination of suitable clauses, wherein the capsicum, the functional mineral compound, and the yeast cell wall are present at a ratio of C:FMC:YCW.
46. The composition of clause 45, any other suitable clause, or any combination of suitable clauses, wherein the ratio of C:FMC:YCW is 1-1000 mg:1-10 g:1-10 g, 100-1000 mg:1-10 g:1-10 g, or 100-500 mg:1-10 g:1-10 g.
47. The composition of clause 45, any other suitable clause, or any combination of suitable clauses, wherein the ratio of C:FMC:YCW is 100 mg:5 g:2 g.
48. The composition of clause 45, any other suitable clause, or any combination of suitable clauses, wherein the ratio of C:FMC:YCW is 300 mg:5 g:2 g.
49. The composition of clause 45, any other suitable clause, or any combination of suitable clauses, wherein the ratio of C:FMC:YCW is 500 mg:5 g:2 g.
50. The composition of clause 45, any other suitable clause, or any combination of suitable clauses, wherein the ratio of C:FMC:YCW is 100 mg:3 g:2 g.
51. The composition of clause 45, any other suitable clause, or any combination of suitable clauses, wherein the ratio of C:FMC:YCW is 300 mg:3 g:2 g.
52. The composition of clause 45, any other suitable clause, or any combination of suitable clauses, wherein the ratio of C:FMC:YCW is 500 mg:3 g:2 g.
53. A formulation comprising i) a composition comprising capsicum, a functional mineral compound, and a yeast cell wall and ii) an animal food.
54. The formulation of clause 53, any other suitable clause, or any combination of suitable clauses, wherein the functional mineral compound comprises an aluminum phyllosilicate clay.
55. The formulation of clause 53, any other suitable clause, or any combination of suitable clauses, wherein the functional mineral compound consists essentially of an aluminum phyllosilicate clay.
56. The formulation of clause 53, any other suitable clause, or any combination of suitable clauses, wherein the functional mineral compound consists of an aluminum phyllosilicate clay.
57. The formulation of clause 53, any other suitable clause, or any combination of suitable clauses, wherein the functional mineral compound comprises bentonite.
58. The formulation of clause 53, any other suitable clause, or any combination of suitable clauses, wherein the functional mineral compound consists essentially of bentonite.
59. The formulation of clause 53, any other suitable clause, or any combination of suitable clauses, wherein the functional mineral compound consists of bentonite.
60. The formulation of clause 53, any other suitable clause, or any combination of suitable clauses, wherein the animal food is a dry animal food.
61. The formulation of clause 60, any other suitable clause, or any combination of suitable clauses, wherein the dry animal food is a starter grain.
62. The formulation of clause 60, any other suitable clause, or any combination of suitable clauses, wherein the dry animal food is a texturized animal food.
63. The formulation of clause 60, any other suitable clause, or any combination of suitable clauses, wherein the dry animal food is a calf starter animal food.
64. The formulation of clause 60, any other suitable clause, or any combination of suitable clauses, wherein the dry animal food is a texturized calf starter animal food.
65. The formulation of clause 60, any other suitable clause, or any combination of suitable clauses, wherein the dry animal food is present in the formulation between about 0.5 lb to about 5 lbs.
66. The formulation of clause 60, any other suitable clause, or any combination of suitable clauses, wherein the dry animal food is present in the formulation between about 0.5 lb to about 3 lbs.
67. The formulation of clause 60, any other suitable clause, or any combination of suitable clauses, wherein the dry animal food is present in the formulation between about 0.5 lb to about 1 lb.
68. The formulation of clause 60, any other suitable clause, or any combination of suitable clauses, wherein the dry animal food is present in the formulation between about 1 lb to about 1.5 lbs.
69. The formulation of clause 60, any other suitable clause, or any combination of suitable clauses, wherein the dry animal food is present in the formulation between about 1.5 lb to about 2 lbs.
70. The formulation of clause 60, any other suitable clause, or any combination of suitable clauses, wherein the dry animal food is present in the formulation between about 2 lb to about 2.5 lbs.
71. The formulation of clause 60, any other suitable clause, or any combination of suitable clauses, wherein the dry animal food is present in the formulation between about 2.5 lb to about 3 lbs.
72. The formulation of clause 60, any other suitable clause, or any combination of suitable clauses, wherein the dry animal food is present in the formulation between about 3 lb to about 3.5 lbs.
73. The formulation of clause 60, any other suitable clause, or any combination of suitable clauses, wherein the dry animal food is present in the formulation between about 3.5 lb to about 4 lbs.
74. The formulation of clause 60, any other suitable clause, or any combination of suitable clauses, wherein the dry animal food is present in the formulation between about 4 lb to about 4.5 lbs.
75. The formulation of clause 60, any other suitable clause, or any combination of suitable clauses, wherein the dry animal food is present in the formulation between about 4.5 lb to about 5 lbs.
76. The formulation of clause 53, any other suitable clause, or any combination of suitable clauses, wherein the animal food is a liquid animal food.
77. The formulation of clause 76, any other suitable clause, or any combination of suitable clauses, wherein the liquid animal food is milk.
78. The formulation of clause 76, any other suitable clause, or any combination of suitable clauses, wherein the liquid animal food is present in the formulation between about 1 quart to about 5 quarts.
79. The formulation of clause 76, any other suitable clause, or any combination of suitable clauses, wherein the liquid animal food is present in the formulation at about 1 quart.
80. The formulation of clause 76, any other suitable clause, or any combination of suitable clauses, wherein the liquid animal food is present in the formulation at about 1.5 quarts.
81. The formulation of clause 76, any other suitable clause, or any combination of suitable clauses, wherein the liquid animal food is present in the formulation at about 2 quarts.
82. The formulation of clause 76, any other suitable clause, or any combination of suitable clauses, wherein the liquid animal food is present in the formulation at about 2.5 quarts.
83. The formulation of clause 76, any other suitable clause, or any combination of suitable clauses, wherein the liquid animal food is present in the formulation at about 3 quarts.
84. The formulation of clause 76, any other suitable clause, or any combination of suitable clauses, wherein the liquid animal food is present in the formulation at about 3.5 quarts.
85. The formulation of clause 76, any other suitable clause, or any combination of suitable clauses, wherein the liquid animal food is present in the formulation at about 4 quarts.
86. The formulation of clause 76, any other suitable clause, or any combination of suitable clauses, wherein the liquid animal food is present in the formulation at about 4.5 quarts.
87. The formulation of clause 76, any other suitable clause, or any combination of suitable clauses, wherein the liquid animal food is present in the formulation at about 5 quarts.
88. The formulation of clause 53, any other suitable clause, or any combination of suitable clauses, wherein the capsicum is present in the composition between about 1 mg to about 1000 mg.
89. The formulation of clause 53, any other suitable clause, or any combination of suitable clauses, wherein the capsicum is present in the composition between about 50 mg to about 750 mg.
90. The formulation of clause 53, any other suitable clause, or any combination of suitable clauses, wherein the capsicum is present in the composition between about 100 mg to about 500 mg.
91. The formulation of clause 53, any other suitable clause, or any combination of suitable clauses, wherein the capsicum is present in the composition at about 100 mg.
92. The formulation of clause 53, any other suitable clause, or any combination of suitable clauses, wherein the capsicum is present in the composition at about 200 mg.
93. The formulation of clause 53, any other suitable clause, or any combination of suitable clauses, wherein the capsicum is present in the composition at about 300 mg.
94. The formulation of clause 53, any other suitable clause, or any combination of suitable clauses, wherein the capsicum is present in the composition at about 400 mg.
95. The formulation of clause 53, any other suitable clause, or any combination of suitable clauses, wherein the capsicum is present in the composition at about 500 mg.
96. The formulation of clause 53, any other suitable clause, or any combination of suitable clauses, wherein the capsicum is present in the composition at about 600 mg.
97. The formulation of clause 53, any other suitable clause, or any combination of suitable clauses, wherein the capsicum is present in the composition at about 700 mg.
98. The formulation of clause 53, any other suitable clause, or any combination of suitable clauses, wherein the capsicum is present in the composition at about 800 mg.
99. The formulation of clause 53, any other suitable clause, or any combination of suitable clauses, wherein the capsicum is present in the composition at about 900 mg.
100. The formulation of clause 53, any other suitable clause, or any combination of suitable clauses, wherein the capsicum is present in the composition at about 1000 mg.
101. The formulation of clause 53, any other suitable clause, or any combination of suitable clauses, wherein the functional mineral compound is present in the composition between about 1 g to about 10 g.
102. The formulation of clause 53, any other suitable clause, or any combination of suitable clauses, wherein the functional mineral compound is present in the composition between about 1 g to about 5 g.
103. The formulation of clause 53, any other suitable clause, or any combination of suitable clauses, wherein the functional mineral compound is present in the composition at about 1 g.
104. The formulation of clause 53, any other suitable clause, or any combination of suitable clauses, wherein the functional mineral compound is present in the composition at about 2 g.
105. The formulation of clause 53, any other suitable clause, or any combination of suitable clauses, wherein the functional mineral compound is present in the composition at about 3 g.
106. The formulation of clause 53, any other suitable clause, or any combination of suitable clauses, wherein the functional mineral compound is present in the composition at about 4 g.
107. The formulation of clause 53, any other suitable clause, or any combination of suitable clauses, wherein the functional mineral compound is present in the composition at about 5 g.
108. The formulation of clause 53, any other suitable clause, or any combination of suitable clauses, wherein the functional mineral compound is present in the composition at about 6 g.
109. The formulation of clause 53, any other suitable clause, or any combination of suitable clauses, wherein the functional mineral compound is present in the composition at about 7 g.
110. The formulation of clause 53, any other suitable clause, or any combination of suitable clauses, wherein the functional mineral compound is present in the composition at about 8 g.
111. The formulation of clause 53, any other suitable clause, or any combination of suitable clauses, wherein the functional mineral compound is present in the composition at about 9 g.
112. The formulation of clause 53, any other suitable clause, or any combination of suitable clauses, wherein the functional mineral compound is present in the composition at about 10 g.
113. The formulation of clause 53, any other suitable clause, or any combination of suitable clauses, wherein the yeast cell wall is present in the composition between about 1 g to about 10 g.
114. The formulation of clause 53, any other suitable clause, or any combination of suitable clauses, wherein the yeast cell wall is present in the composition between about 1 g to about 5 g.
115. The formulation of clause 53, any other suitable clause, or any combination of suitable clauses, wherein the yeast cell wall is present in the composition at about 1 g.
116. The formulation of clause 53, any other suitable clause, or any combination of suitable clauses, wherein the yeast cell wall is present in the composition at about 2 g.
117. The formulation of clause 53, any other suitable clause, or any combination of suitable clauses, wherein the yeast cell wall is present in the composition at about 3 g.
118. The formulation of clause 53, any other suitable clause, or any combination of suitable clauses, wherein the yeast cell wall is present in the composition at about 4 g.
119. The formulation of clause 53, any other suitable clause, or any combination of suitable clauses, wherein the yeast cell wall is present in the composition at about 5 g.
120. The formulation of clause 53, any other suitable clause, or any combination of suitable clauses, wherein the yeast cell wall is present in the composition at about 6 g.
121. The formulation of clause 53, any other suitable clause, or any combination of suitable clauses, wherein the yeast cell wall is present in the composition at about 7 g.
122. The formulation of clause 53, any other suitable clause, or any combination of suitable clauses, wherein the yeast cell wall is present in the composition at about 8 g.
123. The formulation of clause 53, any other suitable clause, or any combination of suitable clauses, wherein the yeast cell wall is present in the composition at about 9 g.
124. The formulation of clause 53, any other suitable clause, or any combination of suitable clauses, wherein the yeast cell wall is present in the composition at about 10 g.
125. The formulation of clause 53, any other suitable clause, or any combination of suitable clauses, wherein the capsicum, the functional mineral compound, and the yeast cell wall are present at a ratio of C:FMC:YCW.
126. The formulation of clause 125, any other suitable clause, or any combination of suitable clauses, wherein the ratio of C:FMC:YCW is 1-1000 mg:1-10 g:1-10 g, 100-1000 mg:1-10 g:1-10 g, or 100-500 mg:1-10 g:1-10 g.
127. The formulation of clause 125, any other suitable clause, or any combination of suitable clauses, wherein the ratio of C:FMC:YCW is 100 mg:5 g:2 g.
128. The formulation of clause 125, any other suitable clause, or any combination of suitable clauses, wherein the ratio of C:FMC:YCW is 300 mg:5 g:2 g.
129. The formulation of clause 125, any other suitable clause, or any combination of suitable clauses, wherein the ratio of C:FMC:YCW is 500 mg:5 g:2 g.
130. The formulation of clause 125, any other suitable clause, or any combination of suitable clauses, wherein the ratio of C:FMC:YCW is 100 mg:3 g:2 g.
131. The formulation of clause 125, any other suitable clause, or any combination of suitable clauses, wherein the ratio of C:FMC:YCW is 300 mg:3 g:2 g.
132. The formulation of clause 125, any other suitable clause, or any combination of suitable clauses, wherein the ratio of C:FMC:YCW is 500 mg:3 g:2 g.
133. A method of improving gastrointestinal health of an animal, said method comprising the step of feeding the animal a composition comprising capsicum, a functional mineral compound, and a yeast cell wall, wherein the composition improves gastrointestinal health in the animal.
134. The method of clause 133, any other suitable clause, or any combination of suitable clauses, wherein improving gastrointestinal health comprises a decrease in frequency of scour events by the animal.
135. The method of clause 133, any other suitable clause, or any combination of suitable clauses, wherein improving gastrointestinal health comprises a decrease in medical intervention from scour events by the animal.
136. The method of clause 133, any other suitable clause, or any combination of suitable clauses, wherein improving gastrointestinal health comprises a decrease in concentration of TNF-α in plasma of the animal.
137. The method of clause 133, any other suitable clause, or any combination of suitable clauses, wherein improving gastrointestinal health comprises an improved innate immune response in the animal.
138. The method of clause 133, any other suitable clause, or any combination of suitable clauses, wherein the animal is a ruminant animal.
139. The method of clause 138, any other suitable clause, or any combination of suitable clauses, wherein the ruminant animal is selected from the group consisting of cattle, goats, sheep, giraffes, American Bison, European bison, yaks, water buffalo, deer, camels, alpacas, llamas, wildebeest, antelope, pronghorn, and nilgai.
140. The method of clause 138, any other suitable clause, or any combination of suitable clauses, wherein the ruminant animal is raised for meat production.
141. The method of clause 138, any other suitable clause, or any combination of suitable clauses, wherein the ruminant animal is raised for dairy production.
142. The method of clause 138, any other suitable clause, or any combination of suitable clauses, wherein the ruminant animal is selected from the group consisting of cattle, buffalo, sheep, and goats.
143. The method of clause 138, any other suitable clause, or any combination of suitable clauses, wherein the ruminant animal is a bovine.
144. The method of clause 138, any other suitable clause, or any combination of suitable clauses, wherein the ruminant animal is a buffalo.
145. The method of clause 138, any other suitable clause, or any combination of suitable clauses, wherein the ruminant animal is a sheep.
146. The method of clause 138, any other suitable clause, or any combination of suitable clauses, wherein the ruminant animal is a goat.
147. The method of clause 138, any other suitable clause, or any combination of suitable clauses, wherein the ruminant animal is a beef cattle.
148. The method of clause 138, any other suitable clause, or any combination of suitable clauses, wherein the ruminant animal is a dairy cattle.
149. The method of clause 138, any other suitable clause, or any combination of suitable clauses, wherein the ruminant animal is a calf.
150. The method of clause 149, any other suitable clause, or any combination of suitable clauses, wherein the calf is a pre-weaned calf.
151. The method of clause 138, any other suitable clause, or any combination of suitable clauses, wherein the ruminant animal is a male.
152. The method of clause 138, any other suitable clause, or any combination of suitable clauses, wherein the ruminant animal is a female.
153. The method of clause 133, any other suitable clause, or any combination of suitable clauses, wherein the composition is a composition of any one of clauses 1-52.
154. The method of clause 133, any other suitable clause, or any combination of suitable clauses, wherein the composition is comprised in the formulation of any one of clauses 53-132.
155. The method of clause 133, any other suitable clause, or any combination of suitable clauses, wherein the composition is administered to the animal one time per day.
156. The method of clause 133, any other suitable clause, or any combination of suitable clauses, wherein the composition is administered to the animal two times per day.
157. The method of clause 133, any other suitable clause, or any combination of suitable clauses, wherein the composition is administered to the animal three times per day.
158. A method of increasing growth performance of an animal, said method comprising the step of feeding the animal a composition comprising capsicum, a functional mineral compound, and a yeast cell wall product, wherein the composition increases growth performance of the animal.
159. The method of clause 158, any other suitable clause, or any combination of suitable clauses, wherein increasing growth performance comprises an increase in average daily gain (ADG) of the animal.
160. The method of clause 159, any other suitable clause, or any combination of suitable clauses, wherein the increase in ADG of the animal is evaluated at weaning of the animal.
161. The method of clause 159, any other suitable clause, or any combination of suitable clauses, wherein the increase in ADG of the animal is evaluated post-weaning of the animal.
162. The method of clause 158, any other suitable clause, or any combination of suitable clauses, wherein the animal is a ruminant animal.
163. The method of clause 162, any other suitable clause, or any combination of suitable clauses, wherein the ruminant animal is selected from the group consisting of cattle, goats, sheep, giraffes, American Bison, European bison, yaks, water buffalo, deer, camels, alpacas, llamas, wildebeest, antelope, pronghorn, and nilgai.
164. The method of clause 162, any other suitable clause, or any combination of suitable clauses, wherein the ruminant animal is raised for meat production.
165. The method of clause 162, any other suitable clause, or any combination of suitable clauses, wherein the ruminant animal is raised for dairy production.
166. The method of clause 162, any other suitable clause, or any combination of suitable clauses, wherein the ruminant animal is selected from the group consisting of cattle, buffalo, sheep, and goats.
167. The method of clause 162, any other suitable clause, or any combination of suitable clauses, wherein the ruminant animal is a bovine.
168. The method of clause 162, any other suitable clause, or any combination of suitable clauses, wherein the ruminant animal is a buffalo.
169. The method of clause 162, any other suitable clause, or any combination of suitable clauses, wherein the ruminant animal is a sheep.
170. The method of clause 162, any other suitable clause, or any combination of suitable clauses, wherein the ruminant animal is a goat.
171. The method of clause 162, any other suitable clause, or any combination of suitable clauses, wherein the ruminant animal is a beef cattle.
172. The method of clause 162, any other suitable clause, or any combination of suitable clauses, wherein the ruminant animal is a dairy cattle.
173. The method of clause 162, any other suitable clause, or any combination of suitable clauses, wherein the ruminant animal is a calf.
174. The method of clause 173, any other suitable clause, or any combination of suitable clauses, wherein the calf is a pre-weaned calf.
175. The method of clause 162, any other suitable clause, or any combination of suitable clauses, wherein the ruminant animal is a male.
176. The method of clause 162, any other suitable clause, or any combination of suitable clauses, wherein the ruminant animal is a female.
177. The method of clause 158, any other suitable clause, or any combination of suitable clauses, wherein the composition is a composition of any one of clauses 1-52.
178. The method of clause 158, any other suitable clause, or any combination of suitable clauses, wherein the composition is comprised in the formulation of any one of clauses 53-132.
179. The method of clause 158, any other suitable clause, or any combination of suitable clauses, wherein the composition is administered to the animal one time per day.
180. The method of clause 158, any other suitable clause, or any combination of suitable clauses, wherein the composition is administered to the animal two times per day.
181. The method of clause 158, any other suitable clause, or any combination of suitable clauses, wherein the composition is administered to the animal three times per day.
182. A method of increasing body weight of an animal, said method comprising the step of feeding the animal a composition comprising capsicum, a functional mineral compound, and a yeast cell wall product, wherein the composition increases body weight of the animal.
183. The method of clause 182, any other suitable clause, or any combination of suitable clauses, wherein the increase in body weight of the animal is evaluated at weaning of the animal.
184. The method of clause 182, any other suitable clause, or any combination of suitable clauses, wherein the increase in body weight of the animal is evaluated post-weaning of the animal.
185. The method of clause 182, any other suitable clause, or any combination of suitable clauses, wherein the animal is a ruminant animal.
186. The method of clause 185, any other suitable clause, or any combination of suitable clauses, wherein the ruminant animal is selected from the group consisting of cattle, goats, sheep, giraffes, American Bison, European bison, yaks, water buffalo, deer, camels, alpacas, llamas, wildebeest, antelope, pronghorn, and nilgai.
187. The method of clause 185, any other suitable clause, or any combination of suitable clauses, wherein the ruminant animal is raised for meat production.
188. The method of clause 185, any other suitable clause, or any combination of suitable clauses, wherein the ruminant animal is raised for dairy production.
189. The method of clause 185, any other suitable clause, or any combination of suitable clauses, wherein the ruminant animal is selected from the group consisting of cattle, buffalo, sheep, and goats.
190. The method of clause 185, any other suitable clause, or any combination of suitable clauses, wherein the ruminant animal is a bovine.
191. The method of clause 185, any other suitable clause, or any combination of suitable clauses, wherein the ruminant animal is a buffalo.
192. The method of clause 185, any other suitable clause, or any combination of suitable clauses, wherein the ruminant animal is a sheep.
193. The method of clause 185, any other suitable clause, or any combination of suitable clauses, wherein the ruminant animal is a goat.
194. The method of clause 185, any other suitable clause, or any combination of suitable clauses, wherein the ruminant animal is a beef cattle.
195. The method of clause 185, any other suitable clause, or any combination of suitable clauses, wherein the ruminant animal is a dairy cattle.
196. The method of clause 185, any other suitable clause, or any combination of suitable clauses, wherein the ruminant animal is a calf.
197. The method of clause 196, any other suitable clause, or any combination of suitable clauses, wherein the calf is a pre-weaned calf.
198. The method of clause 185, any other suitable clause, or any combination of suitable clauses, wherein the ruminant animal is a male.
199. The method of clause 185, any other suitable clause, or any combination of suitable clauses, wherein the ruminant animal is a female.
200. The method of clause 182, any other suitable clause, or any combination of suitable clauses, wherein the composition is a composition of any one of clauses 1-52.
201. The method of clause 182, any other suitable clause, or any combination of suitable clauses, wherein the composition is comprised in the formulation of any one of clauses 53-132.
202. The method of clause 182, any other suitable clause, or any combination of suitable clauses, wherein the composition is administered to the animal one time per day.
203. The method of clause 182, any other suitable clause, or any combination of suitable clauses, wherein the composition is administered to the animal two times per day. 204. The method of clause 182, any other suitable clause, or any combination of suitable clauses, wherein the composition is administered to the animal three times per day.
205. A method of improving intake of food by an animal, said method comprising the step of feeding the animal a composition comprising capsicum, a functional mineral compound, and a yeast cell wall product, wherein the composition improves intake of food by the animal.
206. The method of clause 205, any other suitable clause, or any combination of suitable clauses, wherein the food is a dry food.
207. The method of clause 206, any other suitable clause, or any combination of suitable clauses, wherein the dry food is a starter grain.
208. The method of clause 206, any other suitable clause, or any combination of suitable clauses, wherein the dry food is a texturized animal food.
209. The method of clause 206, any other suitable clause, or any combination of suitable clauses, wherein the dry food is a calf starter animal food.
210. The method of clause 206, any other suitable clause, or any combination of suitable clauses, wherein the dry food is a texturized calf starter animal food.
211. The method of clause 205, any other suitable clause, or any combination of suitable clauses, wherein the food is a liquid food.
212. The method of clause 211, any other suitable clause, or any combination of suitable clauses, wherein the liquid food is milk.
213. The method of clause 205, any other suitable clause, or any combination of suitable clauses, wherein the animal is a ruminant animal.
214. The method of clause 213, any other suitable clause, or any combination of suitable clauses, wherein the ruminant animal is selected from the group consisting of cattle, goats, sheep, giraffes, American Bison, European bison, yaks, water buffalo, deer, camels, alpacas, llamas, wildebeest, antelope, pronghorn, and nilgai.
215. The method of clause 213, any other suitable clause, or any combination of suitable clauses, wherein the ruminant animal is raised for meat production.
216. The method of clause 213, any other suitable clause, or any combination of suitable clauses, wherein the ruminant animal is raised for dairy production.
217. The method of clause 213, any other suitable clause, or any combination of suitable clauses, wherein the ruminant animal is selected from the group consisting of cattle, buffalo, sheep, and goats.
218. The method of clause 213, any other suitable clause, or any combination of suitable clauses, wherein the ruminant animal is a bovine.
219. The method of clause 213, any other suitable clause, or any combination of suitable clauses, wherein the ruminant animal is a buffalo.
220. The method of clause 213, any other suitable clause, or any combination of suitable clauses, wherein the ruminant animal is a sheep.
221. The method of clause 213, any other suitable clause, or any combination of suitable clauses, wherein the ruminant animal is a goat.
222. The method of clause 213, any other suitable clause, or any combination of suitable clauses, wherein the ruminant animal is a beef cattle.
223. The method of clause 213, any other suitable clause, or any combination of suitable clauses, wherein the ruminant animal is a dairy cattle.
224. The method of clause 213, any other suitable clause, or any combination of suitable clauses, wherein the ruminant animal is a calf.
225. The method of clause 224, any other suitable clause, or any combination of suitable clauses, wherein the calf is a pre-weaned calf.
226. The method of clause 213, any other suitable clause, or any combination of suitable clauses, wherein the ruminant animal is a male.
227. The method of clause 213, any other suitable clause, or any combination of suitable clauses, wherein the ruminant animal is a female.
228. The method of clause 205, any other suitable clause, or any combination of suitable clauses, wherein the composition is a composition of any one of clauses 1-52.
229. The method of clause 205, any other suitable clause, or any combination of suitable clauses, wherein the composition is comprised in the formulation of any one of clauses 53-132.
230. The method of clause 205, any other suitable clause, or any combination of suitable clauses, wherein the composition is administered to the animal one time per day.
231. The method of clause 205, any other suitable clause, or any combination of suitable clauses, wherein the composition is administered to the animal two times per day.
232. The method of clause 205, any other suitable clause, or any combination of suitable clauses, wherein the composition is administered to the animal three times per day.
233. The method of any one of clauses 133 to 232, wherein the amount of capsicum administered is between about 1 mg/day/animal to about 1000 mg/day/animal, between about 50 mg/day/animal to about 750 mg/day/animal, or between about 100 mg/day/animal to about 500 mg/day/animal.
234. The method of any one of clauses 133 to 233, wherein, the amount of the functional mineral compound that is administered is between about 1 g/day/animal to about 10 g/day/animal or between about 1 g/day/animal to about 5 g/day/animal.
235. The method of any one of clauses 133 to 233, wherein the amount of the yeast cell wall that is administered is between about 1 g/day/animal to about 10 g/day/animal or between about 1 g/day/animal to about 5 g/day/animal.

### EXAMPLES

### Example 1

### Efficacy of C+FMC+YCW Compositions Versus Comparative C+FMC Products

A total of 150 female calves were utilized in the instant example. Calves were weighed upon arrival to the calf facility and housed in 3 lines of individual calf hutches. Each line of calf hutches was randomly assigned to one of 3 dietary treatment groups in a completely randomized design, resulting in 50 calves per treatment group (Table 1).

TABLE 1
Dietary Treatment Groups

| Treatment | | Inclusion | | Number |
|---|---|---|---|---|
| Group | Additive | Rate/calf/day | | of Calves |
| 1. Control | None | N/A | | 50 |
| 2. C + FMC + YCW | Capsicum | 100 | mg | 50 |
| | Functional Mineral Compound | 5 | g | |
| | Yeast Cell Wall | 2 | g | |
| 3. C + FMC | Capsicum | 100 | mg | 50 |
| | Functional Mineral Compound | 5 | g | |

The instant example was evaluated over 54 days. Calves arrived at the facility at 1-2 days of age and were weaned from milk feedings at 54 days of age. The compositions for each treatment group were combined with milk and were fed to the calves according to the schedule in Table 2. In addition to the milk, calves were offered ad libitum access to a 19% crude protein texturized calf starter and water throughout the trial.

TABLE 2
Milk Feeding Schedule

| Age of calf, days | Amount, qt | Times/day | Solids, lbs/day |
|---|---|---|---|
| 1-3 | 2.60 | 2 | 1.52 |
| 4-16 | 3.00 | 2 | 1.76 |
| 17-45 | 3.75 | 2 | 2.19 |
| 46-48 | 3.00 | 2 | 1.76 |
| 49-51 | 2.60 | 2 | 1.52 |
| 52-54 | 2.60 | 1 | 0.76 |

Calves were weighed on arrival, and at 2, 4, 6, and 8 weeks of life. These data were used to calculate average daily gain (ADG). Fecal scores were documented by 2 independent scorers weekly from weeks 1-4. For fecal scores, 0=normal consistency and 3=watery feces.

Plasma samples were collected from 24 calves randomly selected for the control and C+FMC+YCW treatment groups at arrival and at weaning (day 54). Plasma samples were stored at −20° C. until analyses were performed. Calves consumed 100% of the milk offered. Intake of the texturized calf starter was measured weekly. These data were used to calculate average daily feed intake (ADFI). Incidences of scours that required medical treatment(s) were recorded by personnel caring for calves on a daily basis.

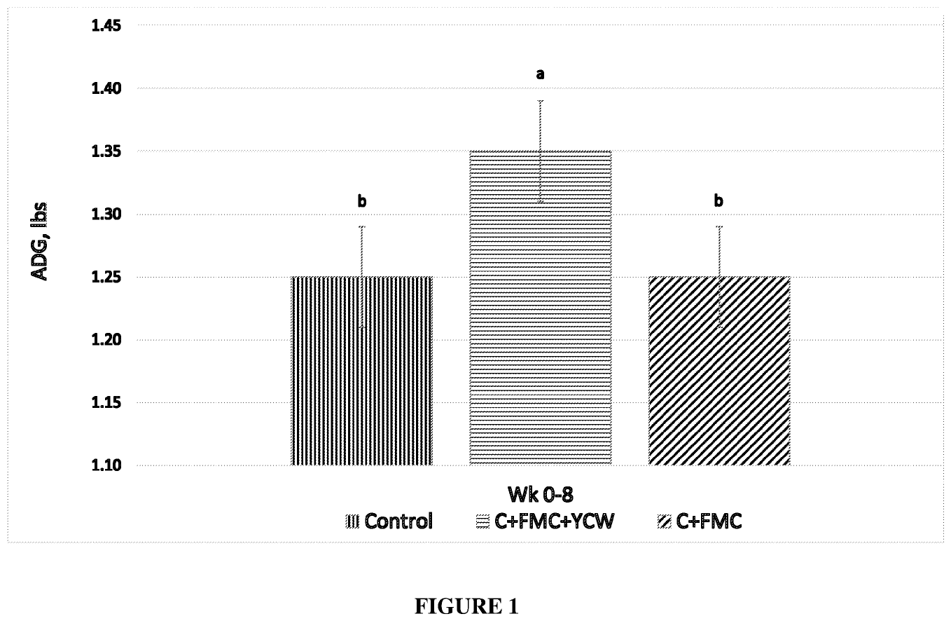

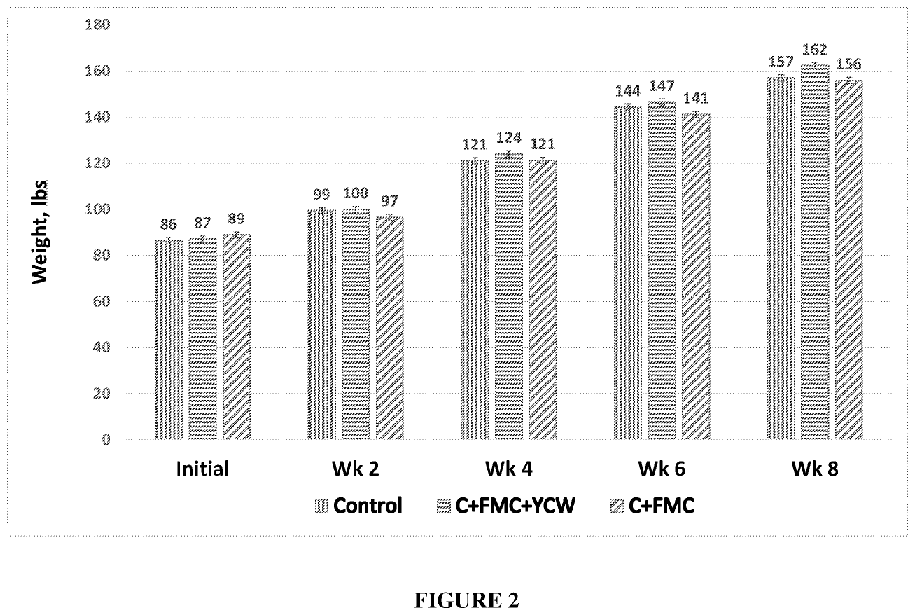

Initial body weight (BW) evaluations were similar for all 3 treatment groups (see FIG. 2). However, ADG was increased in calves by 0.1 lb/day from arrival through weaning when the animals were fed the composition comprising C+FMC+YCW (FIG. 1). For calves fed the comparison composition of C+FMC, ADG was identical to control during this period. The increase in ADG from arrival through weaning for calves fed with C+FMC+YCW resulted in 5 lbs greater BW at weaning compared with control or calves fed with C+FMC (P<0.05; FIG. 2).

Fecal scores and the percentage of calves that received treatment for scouring were reduced for calves fed the composition comprising C+FMC+YWC compared with calves fed control and the comparison composition of C+FMC. Fecal scores and scour treatments are indicators of gastrointestinal health (Table 3).

TABLE 3
Health parameters by dietary treatment

| | | C + FMC + | | | |
|---|---|---|---|---|---|
| Item | Control | YCW | C + FMC | SE | P |
| Fecal Score | 1.38 (a) | 1.25 (b) | 1.31 | 0.035 | 0.005 |
| | | | (a, b) | | |
| Scours, % Calves | 82.3 (a) | 53.5 (b) | 68.3 | 6.91 | 0.03 |
| treated | | | (a, b) | | |

(a, b) Values with different superscripts differ P < 0.05.

TNF-α is a pro-inflammatory cytokine that is activated by the innate immune system in response to pathogens. Lower plasma TNF-α concentrations were observed when animals were fed the composition comprising C+FMC+YWC compared with control (Table 4). Lower circulating TNF-α concentrations indicate that the composition comprising C+FMC+YWC has a positive impact on the innate immune system.

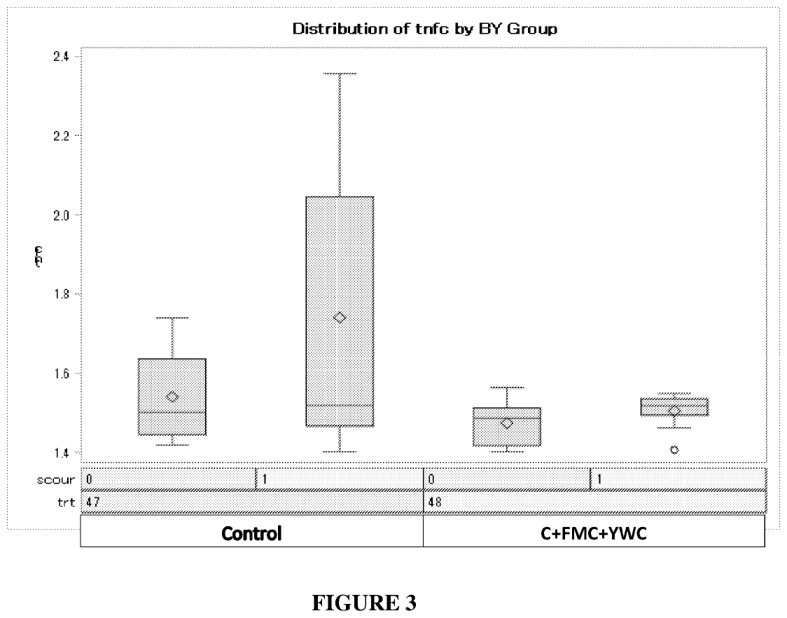

The distribution of TNF-α was also narrower for calves that had a scour event and were fed compositions comprising C+FMC+YWC compared with calves that had a scour event and were fed the control treatment (FIG. 3), which provides supporting evidence for an improved innate immune response when the composition comprising C+FMC+YWC was fed to animals. When capsicum alone was fed to calves at 100 mg/calf/day in a separate trial, there was no impact on plasma TNF-α concentrations (Table 4).

TABLE 4
TNF-α by dietary treatment in two separate trials

| Trial 1 | Control | C + FCM + YCW | SE | P |
|---|---|---|---|---|
| TNF-α initial, pg/mL | 189.73 | 167.26 | 15.03 | N/A |
| TNF-α, pg/mL | 43.19 (a) | 9.89 (b) | 8.67 | 0.0002 |
| Trial 2 | Control 2 | Capsicum alone | | |
| TNF-α initial, pg/mL | 81.07 | 33.31 | 13.13 | N/A |
| TNF-α, pg/mL | 10.17 | 7.45 | 2.40 | 0.45 |

(a, b) Values with different superscripts differ P < 0.05.

Non-esterified fatty acids (NEFA), β-hydroxybutyrate (BHBA), and glucose are metabolites indicative of utilization and intake of dietary energy in the form of fat and carbohydrates. Circulating NEFA and glucose concentrations were increased (P<0.05) with feeding the composition comprising C+FMC+YCW compared with control (Table 5). Further, plasma BHBA concentrations were decreased by 9.68% with feeding the composition comprising C+FMC+YCW compared with control (Table 5). When Functional Mineral Compound and Capsicum were fed individually to calves in a separate trial, there was no effect on circulating NEFA concentrations (Table 6).

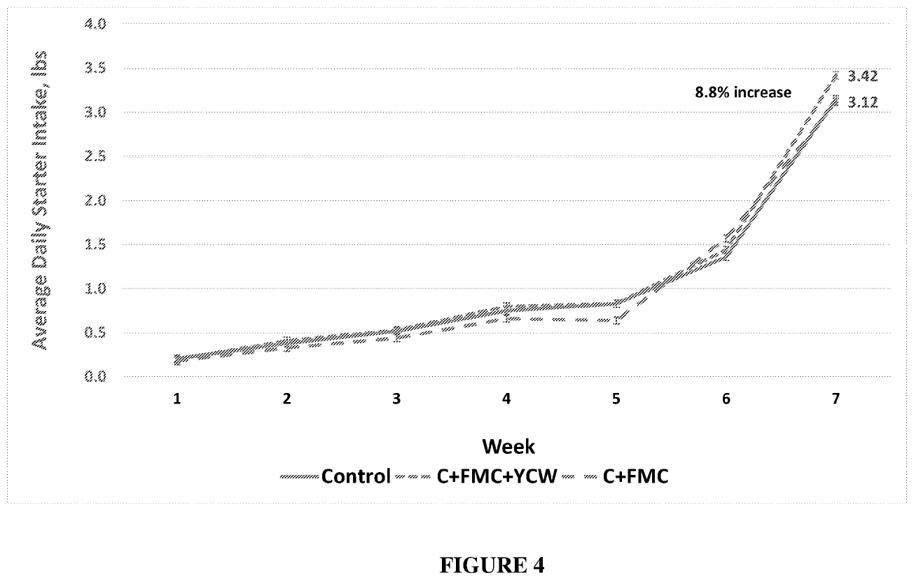

Additionally, glucose and BHBA were both decreased by feeding Functional Mineral Compound and Capsicum individually compared with control (Table 6). Feed intake was also increased by 8.8% for calves fed the composition comprising C+FMC+YCW compared with calves fed control calves fed the comparative composition of C+FMC (FIG. 4).

Together, these data show an improved energy balance during the weaning process for calves that were fed the composition comprising C+FMC+YCW from birth to weaning (Table 5).

TABLE 5
Plasma non-esterified fatty acid, β-hydroxybutyrate,
and glucose concentrations for calves fed control
and C + FMC + YCW

| Item | Control | C + FMC + YCW | SE | P |
|---|---|---|---|---|
| NEFA initial, mEq/L | 343.69 | 347.52 | 18.31 | NA |
| NEFA, mEq/L | 116.81 (b) | 162.75 (a) | 5.03 | <0.001 |
| BHBA initial, μM | 81.511 | 70.369 | 6.84 | NA |
| BHBA, μM | 124.622 | 112.304 | 7.16 | 0.593 |
| Glucose initial, mg/dL | 98.78 | 106.56 | 3.47 | NA |
| Glucose, mg/dL | 93.75 (b) | 112.93 (a) | 4.18 | 0.0017 |

(a, b) Values with different superscripts differ P < 0.05.

TABLE 6
Plasma non-esterified fatty acid, β-hydroxybutyrate, and
glucose concentrations for calves fed FMC and C versus control

| Item | Control | FMC | C | SE | P |
|---|---|---|---|---|---|
| NEFA initial, | 140.31 | 122.35 | 131.69 | 12.85 | NA |
| mEq/L | | | | | |
| NEFA, mEq/L | 130.92 | 117.24 | 131.02 | 10.12 | 0.55 |
| BHBA initial, μM | 102.72 | 68.44 | 103.26 | 9.51 | NA |
| BHBA, μM | 393.59 (a) | 151.19 (b) | 301.19 (b) | 22.01 | <0.0001 |
| Glucose initial, | 101.64 | 157.62 | 166.42 | 5.36 | NA |
| mg/dL | | | | | |
| Glucose, mg/dL | 150.47 (a) | 106.54 (b) | 114.98 (b) | 8.19 | 0.001 |

(a, b) Values with different superscripts differ P < 0.05.

### Example 2

### Efficacy of C+FMC+YCW Compositions Versus Comparative YCW+FMC Products

A total of 250 female calves were utilized in the instant example. Calves were weighed upon arrival to the calf facility and house in 5 lines of individual calf hutches. Each line of calf hutches was randomly assigned to one of 5 dietary treatment groups in a randomized design, resulting in 50 calves per treatment (Table 7).

TABLE 7
Dietary Treatment Groups

| | Feeding rates | | |
|---|---|---|---|
| | | | Capsicum |
| Treatment Group | FMC (g/h/d) | YCW (g/h/d) | (mg/h/d) |
| Control (n = 50) | — | — | — |
| YCW + FMC (n = 50) | 5 | 4 | — |
| C100 + FMC + YWC (n = 50) | 5 | 4 | 100 |
| C300 + FMC + YWC (n = 50) | 5 | 4 | 300 |
| C500 + FMC + YWC (n = 50) | 5 | 4 | 500 |

Dietary treatment groups according to Table 7 were fed through milk for 54 days. Calves arrived at the calf facility at 1-2 days of age and were weaned from milk feedings at 54 days of age. The composition of each treatment group was combined with milk fed to calves according to the schedule of Table 8. In addition to milk, calves were offered ad libitum access to a 19% crude protein texturized calf starter and water throughout the trial.

TABLE 8
Milk feeding schedule

| Age | Amount (qt) | Frequency | Solids/day |
|---|---|---|---|
| 1-3 | 2.6 | 2 | 1.52 |
| 4-16 | 3 | 2 | 1.76 |
| 17-45 | 3.75 | 2 | 2.19 |
| 46-48 | 3 | 2 | 1.76 |
| 49-51 | 2.6 | 2 | 1.52 |
| 52-54 | 2.6 | 1 | 0.76 |

Calves were weighed upon arrival, and at 2, 4, 6, and 8, 9 and 12 weeks of life. Calves consumed 100% of the milk offered. The data collected were used to calculate ADG. Incidences of scours that required medical treatment(s) were recorded by personnel caring for calves on a daily basis.

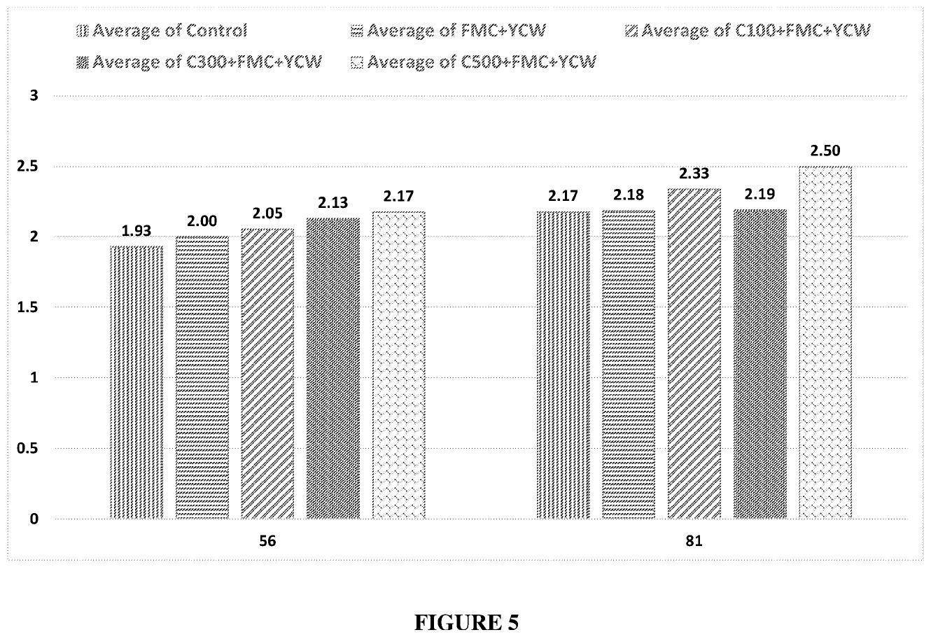

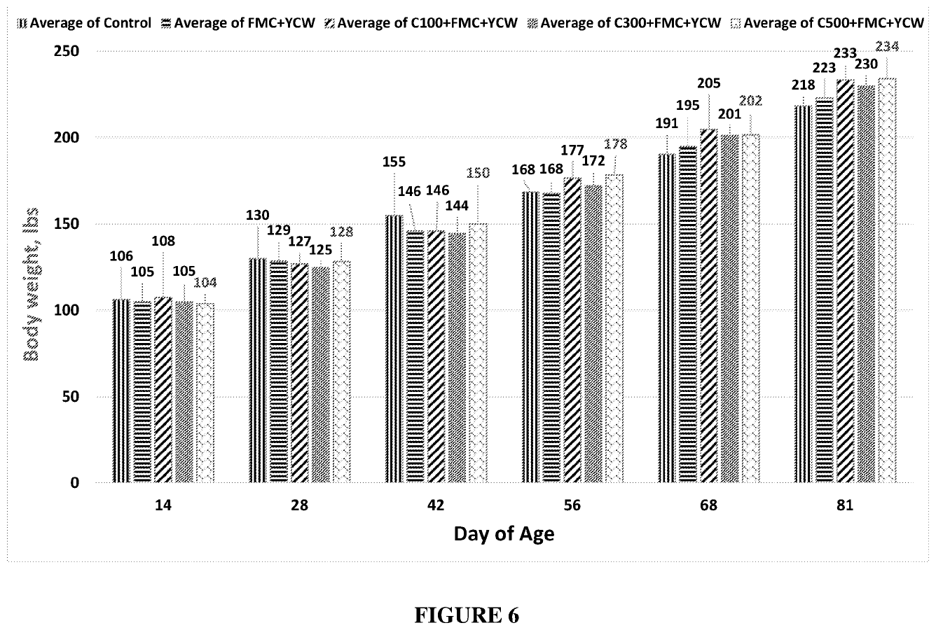

Average daily gain (ADG) for the weighing intervals between 42 and 56 and also between 68 and 81 days of age were greater for the calves fed the compositions comprising C+FMC+YWC compared with calves fed the control and compositions of FMC+YCW (FIG. 5). This improvement in ADG, both pre- and post-weaning, resulted in increased body weight (BW) for calves fed the compositions comprising C+FMC+YWC compared with calves fed the control and compositions of FMC+YCW (Table 9 and FIG. 6; P<0.05).

Weaning is a stressful event for calves as they move from milk-based diets to a solid feed diet. Post-weaning relocation to group pens are additional stressful events for calves. These two stress events close together often result in growth loss or growth stagnation. However, the instant example demonstrates that feeding compositions comprising C+FMC+YCW increased growth in calves during these stressful periods.

TABLE 9
Body weight (lbs) by dietary treatment

| | | | C100 + FMC + | C300 + FMC + | C500 + FMC + |
|---|---|---|---|---|---|
| Day | Control | FMC + YCW | YCW | YCW | YCW |
| 14 | 106.21 | 105.15 | 107.52 | 104.95 | 103.86 |
| 28 | 129.83 | 128.62 | 127.03 | 124.6 | 128.32 |
| 42 | 154.98 | 145.86 | 145.81 | 144.45 | 150.09 |
| 56 | 168.49 | 167.6 | 176.59 | 172.12 | 178.34 |
| 68 | 190.52 | 194.67 | 204.74 | 201.37 | 201.74 |
| 81 | 218.47b | 222.95b | 233.42a | 229.88a | 234.18a |

a, b Values with different superscripts differ P < 0.05.

### Example 3

### Efficacy of C+FMC+YCW Compositions in Combination with Animal Food Improves Growth Performance and Calf Health

A total of 300 female calves were utilized in the instant example. Calves were weighed upon arrival to the calf facility and housed in 6 lines of 50 individual calf hutches. Two lines of calf hutches were randomly assigned to one of 3 dietary treatment groups in a randomized design, resulting in 100 calves per treatment (Table 10).

TABLE 10
Dietary Treatment Groups

| | | | Functional | | |
|---|---|---|---|---|---|
| | Yeast Cell | | Mineral | | Number |
| Treatment | Wall | Capsicum | Compound | | of |
| Groups | g/calf/d | mg/calf/d | g/calf/d | Delivery | calves |
| Control | — | — | — | Placebo in milk | 100 |
| C + FCM + YCW | 2 | 100 | 3 | Milk | 100 |
| C + FCM + YCW | 2 | 100 | 3 | Dry Feed | 100 |

Dietary treatment groups were fed through the milk or supplemented to texturized calf starter from arrival to for 84 days. Calves arrived at the calf facility at 1-2 days of age and were weaned from milk feedings at 52 days of age. All calves received a common milk feeding schedule (Table 11), and all calves were offered ad libitum access to a 19% crude protein texturized calf starter and water throughout the trial.

TABLE 11
Milk Feeding Schedule

| Age | Amount (qt) | Frequency | Solids, lbs/day |
|---|---|---|---|
| 1-14 | 2.9 | 2 | 1.63 |
| 15-26 | 3.3 | 2 | 1.86 |
| 27-36 | 2.9 | 2 | 1.63 |
| 37-44 | 2.5 | 2 | 1.41 |
| 45-52 | 2.5 | 1 | 0.70 |

For calves receiving compositions comprising C+FCM+YCW via milk, the compositions were combined with the milk at time of delivery. For calves receiving compositions comprising C+FCM+YCW via dry feed, the compositions were provided on top of the texturized calf starter for calves on the dry feed treatment after delivery of feed into the bucket. Post-weaning, all calves fed compositions comprising C+FCM+YCW continued to receive the compositions via dry feed in both the individual calf hutch and when they were moved to group housing (Table 12). Incidences of scours that required medical treatment(s) were recorded by personnel caring for calves on a daily basis.

TABLE 12
Delivery of dietary treatment groups by day of age

| | | | Product | Product into |
|---|---|---|---|---|
| Treatment | Age | | into | Grain |
| Group | (days) | Housing | Milk | Pellet |
| Control | 1-70 | Calf hutches | Placebo | Placebo |
| | 70-84 | Group pens | N/A | Placebo |
| C + FMC + YCW | 1-70 | Calf hutches | Treatment | Placebo |
| in milk | 70-84 | Group Pens | N/A | Treatment |
| C + FMC + YCW | 1-70 | Calf hutches | Placebo | Treatment |
| in dry feed | 70-84 | Group Pens | N/A | Treatment |

Calves were weighed upon arrival, and at 4, 6, 8, 10 and 12 weeks of life. Fecal scores were documented by 2 independent scorers weekly from weeks 1-4. For fecal scores 0=normal consistency and 3=watery feces. Calves consumed 100% of the milk offered. Intake of the texturized calf starter was measured weekly. The data collected was used to calculate ADG and ADFI (Table 13). Incidences of scours were recorded by personnel caring for calves on a daily basis.

TABLE 13
Body weight, ADG, starter intake, and scour incidences for 3 dietary treatment groups

| | | | C + FMC + YCW | | C + FMC + YCW | | | |
|---|---|---|---|---|---|---|---|---|
| Item | Control | | Milk | | Starter | | SE | P |
| Body Weight, lbs | 160.1 | (b) | 169.6 | (a) | 169.1 | (a) | 1.63 | 0.0001 |
| ADG by weighing | 1.36 | (b) | 1.62 | (a) | 1.62 | (a) | 0.05 | 0.004 |
| period, lbs | | | | | | | | |
| ADG from arrival, lbs | 1.25 | (b) | 1.41 | (a) | 1.42 | (a) | 0.04 | 0.006 |
| Starter Intake, lbs | 1.64 | (b) | 1.85 | (a) | 1.64 | (b) | 0.06 | 0.02 |
| Scours, % of calves | 31 | (a) | 20 | (b) | 14 | (b) | 0.50 | 0.0003 |

(a, b) Values with different superscripts differ P < 0.05.

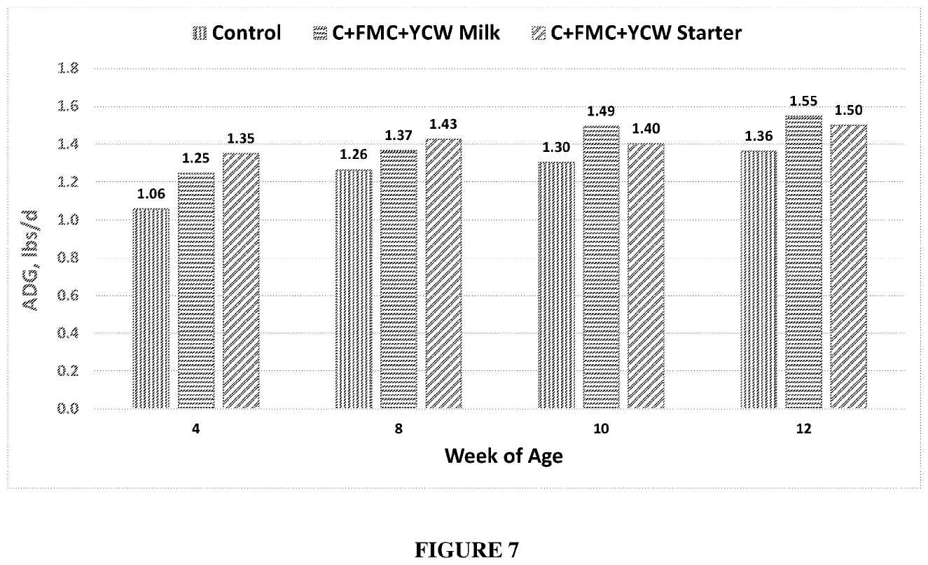

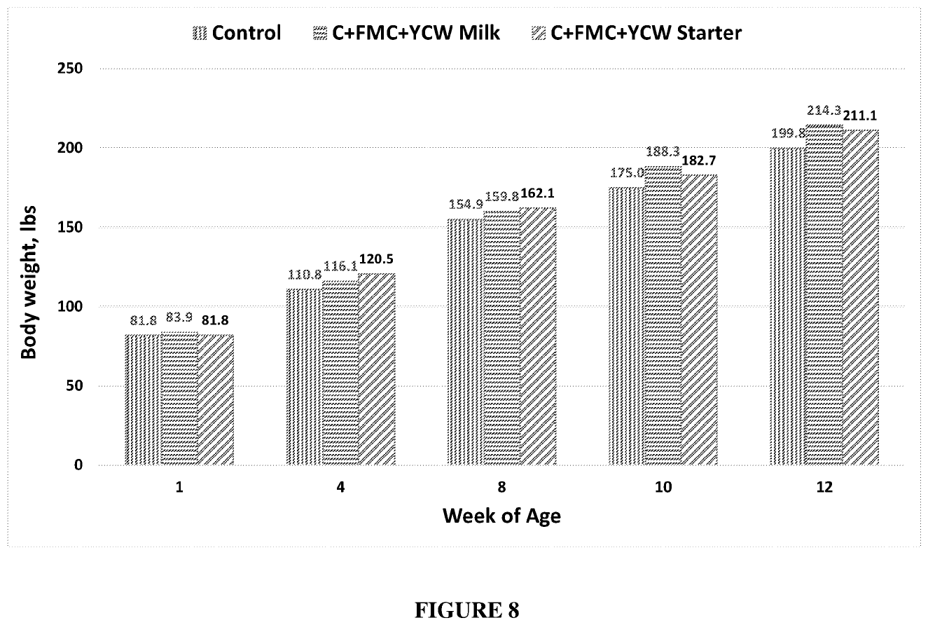

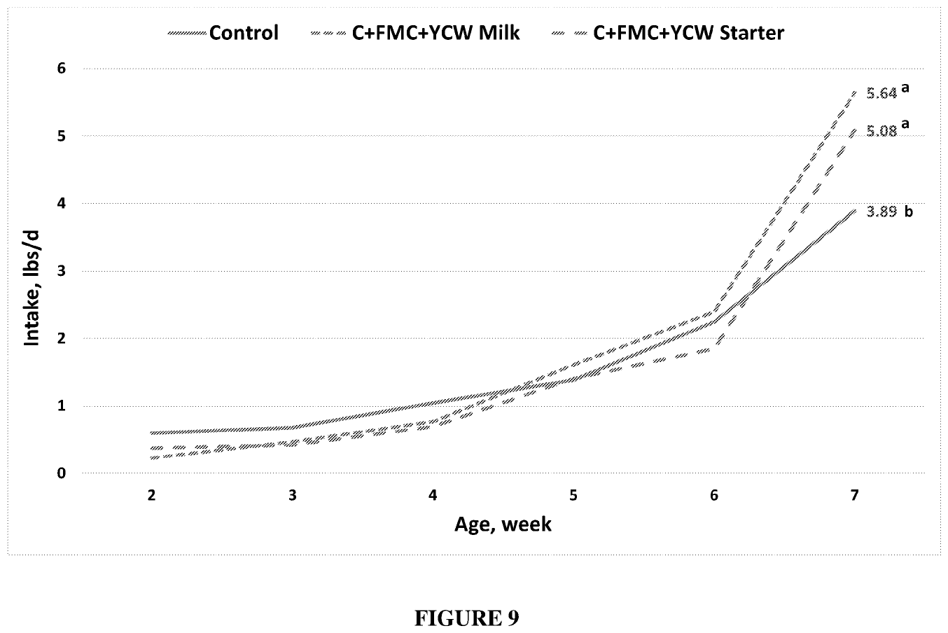

Calves fed the compositions comprising C+FMC+YCW, via both milk and dry feed, demonstrated increased ADG compared with calves fed control from weeks 4-12 (FIG. 7). The improvement in ADG resulted in heavier calves at weaning and post-weaning (FIG. 8). Intake of dry feed was also improved for calves fed compositions comprising C+FMC+YCW via both milk and dry feed compared with calves fed control (FIG. 9). Upon weaning, calf health and growth are believed to be dependent upon consumption of dry feed. Calves that have increased consumption of dry feed prior to weaning are better prepared for the weaning transition.

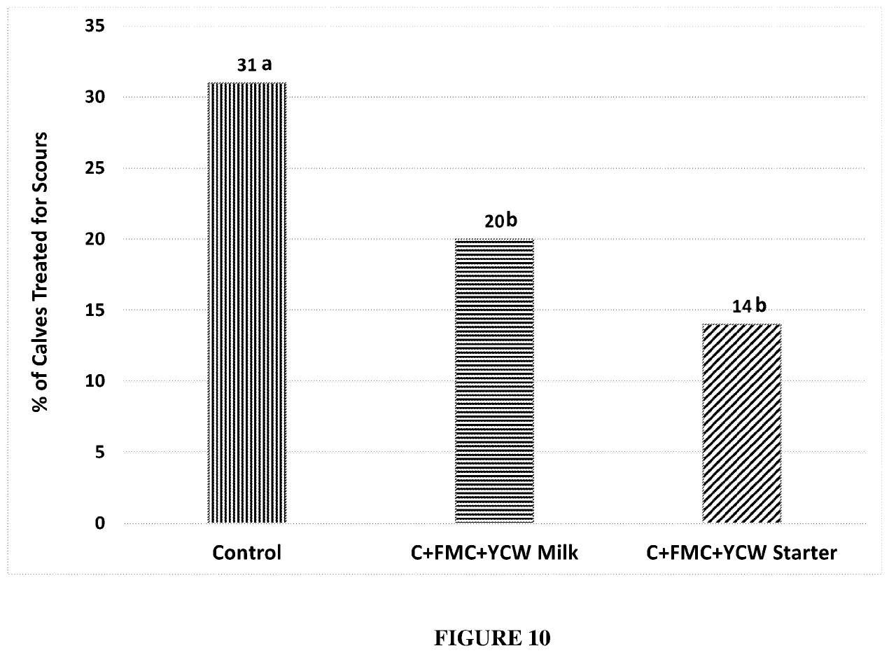

Total incidences of scours were reduced when compositions comprising C+FMC+YCW was fed to calves via both milk and dry feed compared with calves fed control (FIG. 10). Scouring or diarrhea is a response to stress and/or exposure to gastrointestinal pathogens. Therefore, feeding compositions comprising C+FMC+YCW via either milk or dry feed in early life appears to improve intestinal health in calves by reducing incidences of loose and watery stool that may require medical intervention and treatment.

1. A composition comprising capsicum, a functional mineral compound, and a yeast cell wall.

2. The composition of claim 1, wherein the functional mineral compound comprises an aluminum phyllosilicate clay.

3. The composition of claim 1, wherein the functional mineral compound comprises bentonite.

4.-9. (canceled)

10. The composition of claim 1, wherein the capsicum, the functional mineral compound, and the yeast cell wall are present at a ratio of C:FMC:YCW, and wherein the ratio of C:FMC:YCW is 1-1000 mg:1-10 g:1-10 g, 100-1000 mg:1-10 g:1-10 g, or 100-500 mg:1-10 g:1-10 g.

11.-26. (canceled)

27. A method of improving gastrointestinal health of an animal, said method comprising the step of feeding the animal a composition comprising capsicum, a functional mineral compound, and a yeast cell wall product, wherein the composition improves gastrointestinal health in the animal.

28. The method of claim 27, wherein improving gastrointestinal health comprises a decrease in frequency of scour events by the animal.

29. The method of claim 27, wherein improving gastrointestinal health comprises a decrease in medical intervention from scour events by the animal.

30. The method of claim 27, wherein improving gastrointestinal health comprises a decrease in concentration of TNF-α in plasma of the animal.

31. The method of claim 27, wherein improving gastrointestinal health comprises an improved innate immune response in the animal.

32. A method of increasing growth performance of an animal, said method comprising the step of feeding the animal a composition comprising capsicum, a functional mineral compound, and a yeast cell wall product, wherein the composition increases growth performance of the animal.

33. The method of claim 32, wherein increasing growth performance comprises an increase in average daily gain (ADG) of the animal.

34. The method of claim 32, wherein the increase in ADG of the animal is evaluated at weaning of the animal.

35. The method of 32, wherein the increase in ADG of the animal is evaluated post-weaning of the animal.

36.-49. (canceled)

50. The method of claim 27, wherein the amount of capsicum administered is between about 1 mg/day/animal to about 1000 mg/day/animal, between about 50 mg/day/animal to about 750 mg/day/animal, or between about 100 mg/day/animal to about 500 mg/day/animal.

51. The method of claim 27, wherein, the amount of the functional mineral compound that is administered is between about 1 g/day/animal to about 10 g/day/animal or between about 1 g/day/animal to about 5 g/day/animal.

52. The method of claim 27, wherein the amount of the yeast cell wall that is administered is between about 1 g/day/animal to about 10 g/day/animal or between about 1 g/day/animal to about 5 g/day/animal.

53. The method of claim 27, wherein the composition comprises the composition of claim 1.

54. The method of claim 32, wherein the amount of capsicum administered is between about 1 mg/day/animal to about 1000 mg/day/animal, between about 50 mg/day/animal to about 750 mg/day/animal, or between about 100 mg/day/animal to about 500 mg/day/animal.

55. The method of claim 32, wherein, the amount of the functional mineral compound that is administered is between about 1 g/day/animal to about 10 g/day/animal or between about 1 g/day/animal to about 5 g/day/animal.

56. The method of claim 32, wherein the amount of the yeast cell wall that is administered is between about 1 g/day/animal to about 10 g/day/animal or between about 1 g/day/animal to about 5 g/day/animal.
